(19) United States
(12) Patent Application Publication
(10) Pub. No.: US 2009/0255535 A1
(43) Pub. Date: Oct. 15, 2009

(54) DROPLET COLLECTION DEVICES AND METHODS TO DETECT AND CONTROL AIRBORNE COMMUNICABLE DISEASES UTILIZING RFID

(76) Inventor: Steve H. Kanzer, Ann Arbor, MI (US)

Correspondence Address: HAYES SOLOWAY P.C., 3450 E. SUNRISE DRIVE, SUITE 140, TUCSON, AZ, 85718, US

(21) Appl. No.: 12/486,679

(22) Filed: Jun. 17, 2009

Related U.S. Application Data
(62) Division of application No. 11/627,329, filed on Jan. 25, 2007.
(60) Provisional application No. 60/762,201, filed on Jan. 25, 2006.

(51) Int. Cl. A62B 7/10; A61B 5/01; G01N 21/55; H01L 31/00; G01D 21/00

(52) U.S. Cl. 128/206.14; 128/206.15; 600/549; 128/206.12; 356/445; 250/330; 116/206; 128/898

(57) ABSTRACT

A filtering face mask that comprises: (a) a mask body; and (b) at least one diagnostic device for identifying whether the wearer of the mask is infected with a contagious disease.

### CROSS REFERENCE TO RELATED APPLICATION

This application claims priority to U.S. Provisional Application Ser. No. 60/762,201, filed Jan. 25, 2006.

### FIELD OF THE INVENTION

The present invention relates to droplet collection devices and methods to detect and control airborne communicable diseases in humans and/or animals utilizing RFID. The present invention has particular applicability to expiratory droplet collection devices and functions that may be incorporated with or into face masks to detect and control outbreaks of airborne communicable diseases through the identification, tracking and quarantine of potentially infectious humans and/or animals, utilizing RFID (Radio Frequency Identification) technology and other automatic identification systems.

### BACKGROUND OF THE INVENTION

Despite tremendous advances in medical science and technologies available, society's preparedness and procedures to control outbreaks of serious airborne communicable diseases have not advanced considerably from the physical quarantining procedures developed centuries ago. Although a substantially greater number of therapeutic options are now available to treat affected persons, the risk of outbreak of a new highly communicable, serious or life threatening disease that is resistant or difficult to treat with existing therapies is always present. Recent epidemics such as Avian Flu and Severe Acute Respiratory Syndrome (SARS) have confirmed that the speed and popularity of international travel can rapidly transform local outbreaks into potential global pandemics. Intermingling in high density environments, such as, mass transportation, workplaces, hospitals, schools, malls, aircraft, restaurants and other places of congregation, is common to our daily lives and creates an environment in which airborne pathogens can and do spread quickly and insidiously. Certain pathogens may also be spread to humans by contact with infected livestock, fowl, pets, or other animals. Furthermore, advances in genetic engineering make possible the threat of intentionally engineered pathogens as agents of bio-terror that are both highly communicable and associated with poor rates of recovery.

In addition to the human cost of a potential global pandemic, suspected outbreaks can also wreak economic havoc on local economies, as people fear infection and possible quarantine. During the SARS scare of 2003, Toronto utilized a procedure of individually screening arriving international passengers utilizing digital ear thermometers to detect fever. Some Asian countries continue to use such a procedure as well as adopting similar procedures like thermal image scanning. In retrospect, the Toronto procedure was criticized for failing to identify infected, contagious persons due to the fact that exhibition of fever occurs late in the disease. Tests producing a false positive or false negative result are therefore quite common and pose a major drawback to these types of screening procedures. Furthermore, the practicality of this approach limited its widespread use beyond airports given the labor intensive nature of the procedure.

The SARS outbreak in China and Taiwan in 2003 demonstrated some additional flaws in current procedures for controlling and treating outbreaks of serious communicable diseases. In those cases, an inadequate approach was taken to protecting the hospital and healthcare workers upon whom society must inevitably rely to treat the infected and sick. As a result, doctors and nurses lost their lives and entire hospitals were quarantined. In addition, authorities had a difficult time locating potentially infected persons, most likely out of these persons' fear of being quarantined on the basis of an over-inclusive arbitrary order based not upon evidence of infection or disease, but on the basis of location, time and place instead. Had the same scenario played out in a country such as the United States that features an individual-centered culture with more liberal standards and less fear of severe punishment for failure to comply with quarantine orders, it is difficult to predict the reaction of individuals and families. Such persons could elect to flee an infected area in advance of potential quarantine, thereby undermining the objectives of controlling spread of disease.

Disposable and non-disposable face masks have been in use for many years to limit the transmission of communicable diseases capable of transmission via airborne means. In the medical field, early masks were used to prevent contamination and resulting infection of or by patients, particularly during surgery. In recent years, there has also been an increased awareness and concern for preventing contamination and infection of the public by airborne pathogens. Current guidelines and recommendations of the Centers for Disease Control (CDC), for example, recommend the use of face masks to control influenza when suboptimal immunization of the public could increase the frequency of influenza infection.

Human influenza is transmitted from person to person primarily via virus-laden large droplets (particles>5 μm in diameter) that are generated when infected persons cough or sneeze. These large droplets can then be directly deposited onto the mucosal surfaces of the upper respiratory tract of susceptible persons who are near (i.e., within 3 feet) the droplet source. Transmission also may occur through direct and indirect contact with infectious respiratory secretions or infectious expiratory droplets or airborne droplet nuclei.

A combination of infection control strategies is recommended to decrease transmission of influenza in health-care settings. These include placing influenza patients in private rooms when possible, and having health-care personnel wear masks for close patient contact (i.e., within 3 feet) and gowns and gloves if contact with expiratory droplets is likely. The use of surgical or procedure masks by infectious patients may help contain their expiratory droplets and limit exposure to others. Likewise, when a patient is not wearing a mask, as when in an isolation room, having health-care personnel wear masks for close contact with the patient may prevent nose and mouth contact with respiratory droplets. In the United States, disposable surgical and procedure masks have been used widely in healthcare settings to prevent exposure to respiratory infections, but they have not been used commonly in community settings (e.g. schools, businesses, and public gatherings).

The standard protective face mask of the prior art is a disposable, paper mask and generally falls into two categories: molded, cup-shaped masks and fold-flat masks. Molded cup-shaped masks offer the advantage of having a firmly constructed mask body that is spaced from the wearer's face. They may be formed from one or more layers of air-permeable material. Many of the “N95” masks recommended by the CDC for maximum protection of health care workers during outbreaks of diseases such as Avian Influenza and SARS are the molded, cup-shaped type. Examples of such masks are described in U.S. Pat. Nos. 4,536,440; 4,807,619; 4,850,347; 5,307,796 and 5,374,458. Fold-flat masks are constructed to fold-flat for storage and to open out to provide a cup-shaped air chamber over the mouth and nose of the wearer during use. These masks may also be formed from layers of air permeable material. Examples of fold-flat masks are described in U.S. Pat. Nos. 5,322,061; 5,020,533; 4,920,960 and 4,600,002.

A face mask desirably covers a wearer's nose and mouth and even more desirably, a portion of the wearer's face, i.e., cheeks, jaw, chin, and so forth. Disposable face masks are preferably light-weight and inexpensive. Many face masks have ties on each side, while some face masks have one or more elastic bands or straps that extend from one side of the mask to the other to secure the mask to the wearer's head. The mask may also incorporate other attached components including valves, nose clips, and face shields, all of which are well known in the art.

In non-healthcare settings, face masks are recommended whenever symptomatic persons leave home and are in public places to limit the risk of transmission to others in close contact. Face masks are commonly used by the public in the event of suspected or potential outbreaks of serious airborne communicable disease. Face masks in use to date generally serve to protect the wearer from airborne infectious diseases as well as to protect others from exposure to infectious aerosols and particles that the wearer may potentially transmit. The mandatory use of face masks in public areas such as hospitals, mass transit systems and other places of congregation as well as poultry processing facilities is sometimes mandated by health authorities to limit the spread of outbreaks of potentially serious diseases capable of airborne transmission. In addition to ordering the wearing of face masks, health authorities have historically taken additional precautions by ordering the quarantine or exclusion of persons considered at high risk of infection based upon their presence in areas considered at high risk of infection. Faced with the influenza pandemic of 1918, for example, the state of New York issued an order prohibiting congregation of citizens in public areas. During the SARS outbreak in China, that occurred between November, 2002 and July, 2003, the Chinese government quarantined residents of certain areas to prevent the potential spread of the disease. In 2003, Toronto health authorities mandated a procedure in which arriving airline passengers were individually screened for potential SARS infection by means of an electronic thermometer placed in the ear of each passenger upon arrival. Also in 2003, thermal imaging scans were instituted to screen passengers at Chiangi airport in Singapore. Such measures may have been more widely applied had it not been for the laborious process of testing persons one-by-one as well as the associated inconvenience and delays imposed upon the tested subjects.

In view of the above, a need exists for an efficient and reliable systems, i.e., methods and devices to rapidly and effectively identify and control outbreaks of serious airborne communicable diseases while minimizing the socioeconomic burden and counter-productive panic of potentially infected persons in fear of overly broad quarantine procedures, such as those based upon location or travel history. Accordingly, an object of the present invention is to provide novel devices and methods to rapidly collect, identify, analyze, track, control, accurately quarantine and improve treatment for outbreaks or suspected outbreaks of highly communicable diseases, although the invention is not necessarily limited to use for diseases having serious pandemic potential.

A further object of the present invention is to provide a diagnostic device which is capable of conveniently and quickly identifying communicable persons by the collection and analysis of bio-samples so that contagious persons may be separated from non-contagious persons. A further object of the invention is to provide a biosampling device that it will not interfere with the personal protection afforded by the continuous wear of a face mask.

A further object of the present invention is to provide methods of use of the diagnostic device to collect personal information from infectious and non-infectious people and thereby track outbreaks across a population. Another object of the present invention is to improve compliance with recommended procedures by providing evidence of a person's improper use or lack of use of a personal protective mask, thereby reducing the potential number of persons unnecessarily subject to quarantine or other restrictions.

All patents and published patent applications referenced herein are hereby incorporated by reference in their entirety.

### SUMMARY OF THE INVENTION

The present invention comprises a “smart” respiratory filtering face mask that accomplishes the traditional functions of prior art face masks such as providing a filtering device for protecting the wearer of the mask from exposure to external airborne communicable pathogens, and providing a filtering device for protecting other persons from exposure to airborne communicable pathogens potentially expired by the wearer of the mask. In addition to these traditional filtering and protection functions, the present invention further comprises at least one diagnostic device to identify wearers that may potentially be infectious and contagious and therefore pose a threat of infecting others. The present invention also provides governments, health authorities, hospitals and others with efficient, convenient and cost effective methods to identify and track potentially serious outbreaks of communicable diseases.

### BRIEF DESCRIPTION OF THE DRAWINGS

FIG. 1 is a rear view of a fold-flat, filtering face mask 1, that is fitted with a first embodiment of a unilateral expiratory droplet collection strip 2, in accordance with the present invention.

FIG. 2A is a front view of a unilateral expiratory droplet collection strip 2, in accordance with the present invention.

FIG. 2B is a rear view of a unilateral expiratory droplet collection strip 2, in accordance with the present invention.

FIG. 3 is a top, cross-sectional view of a semi-folded unilateral expiratory droplet collection strip 2, in accordance with the present invention.

FIG. 4 is a sectional side view of a molded, cup-shaped mask 27 that is fitted with a first embodiment of a unilateral expiratory droplet collection strip 2, in accordance with the present invention.

FIG. 5 is a copy of a sheet 33 of uniquely identified, computer-generated, mass-produced, pre-printed, bar-coded unilateral expiratory droplet collection strips 2, in accordance with the present invention.

FIG. 6 is a perspective view of a molded, cup-shaped filtering face mask 34 which is fitted with an exhalation valve 35 having a detachable filtering cartridge 36, in accordance with the present invention.

FIG. 7 is a perspective view of a molded, cup-shaped filtering face mask 34, which is fitted with an immunoassay device 37, in accordance with the present invention.

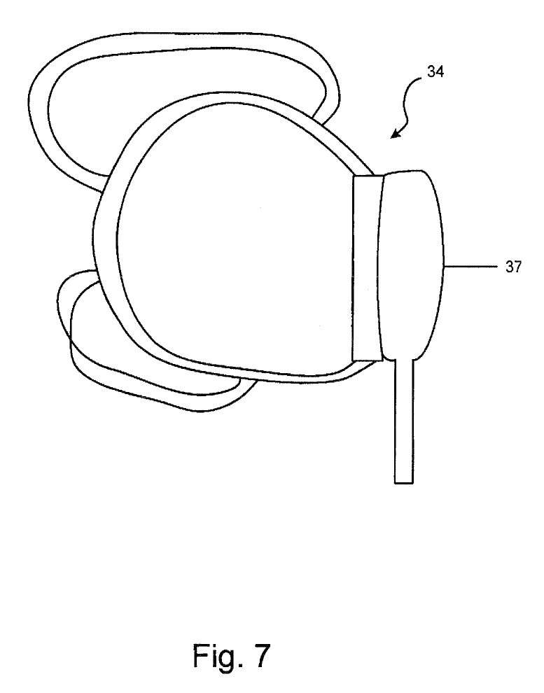

FIG. 8 is a perspective view of a first embodiment of an immunoassay device 37 which comprises a fluid-collection chamber 37B, as shown in FIG. 7.

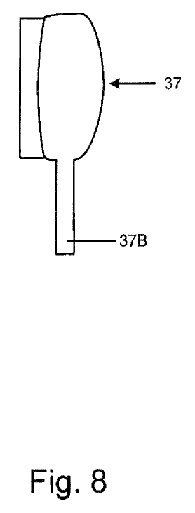

FIG. 9 is a rear view of a filtering face mask 34, which is fitted with a first embodiment of a bio-sample collection apparatus 38, in accordance with the present invention.

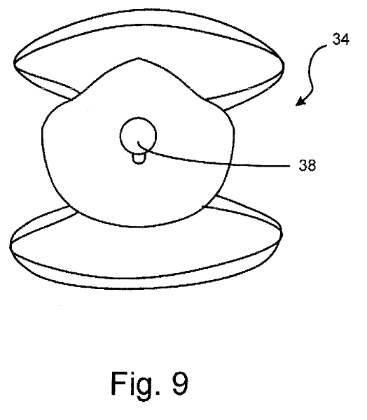

FIG. 10 is a perspective view of a filtering face mask 34, which is fitted internally with an absorbent bio-sample collection swab 39 which may be detached from mask 34 by pulling tab 40 in a downward direction, in accordance with the present invention.

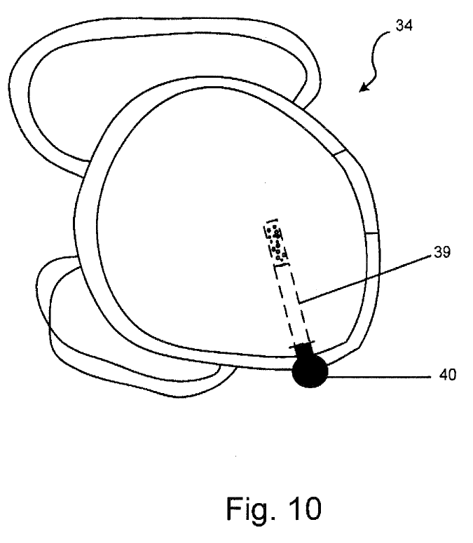

FIG. 11 is a sectional side view of a filtering face mask 34, which is fitted internally with an absorbent bio-sample collection swab 39, which may be detached from mask 34 by pulling tab 40 in a downward direction, in accordance with the present invention.

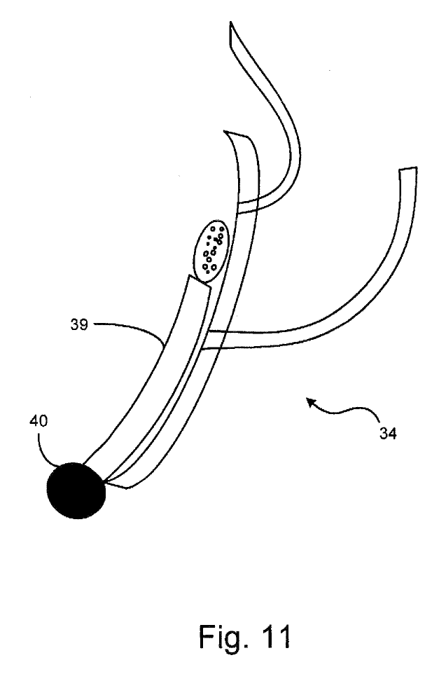

FIG. 12 is a view of one layer of a unilateral expiratory droplet collection strip 2, 11 which is fitted with an Avery Dennison, Inc. AD-220 RFID tag inlay 41 that is affixed to the layer, in accordance with the present invention.

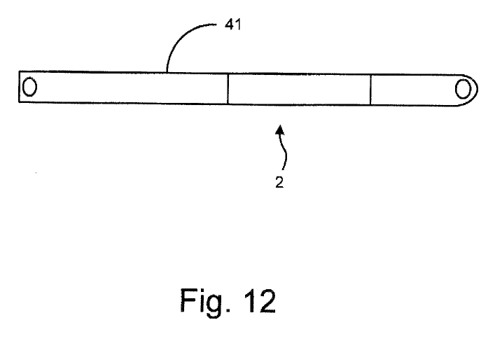

### DETAILED DESCRIPTION OF THE INVENTION

Reference will now be made in detail to the presently preferred embodiments of the invention, one or more examples of which are illustrated in the drawings. Each example is provided by way of explanation of the invention and is not meant as a limitation of the invention. For example, features illustrated and described as part of one embodiment or figure can be used on another embodiment or figure to yield yet another embodiment. It is intended that the present invention include such modifications and variations.

### DEFINITIONS

In reference to the invention, the following terms are defined as set forth below.

As used herein, “airborne” refers to infectious agents that may be transmitted from persons or animals or environments by either “droplet infection” via the transmission of infectious droplets, or by “airborne transmission” via the transmission of infectious “airborne droplet nuclei”.

As used herein, the phrase “airborne droplet nuclei” refers to small-particle residue (5 μm or smaller in size) of evaporated droplets that may remain suspended in the air for long periods of time. Examples of serious illnesses which are transmitted by airborne droplet nuclei include, but are not limited to, measles, varicella-zoster virus infections, legionella, disseminated zoster, tuberculosis, canine influenza, psittacosis, inclusion body disease of boid snakes, foot-and-mouth disease (FMD), SARS, and avian influenzas including influenza A (H5N1) and H5N9.

As used herein, the phrase “airborne transmission” refers to the dissemination of either airborne droplet nuclei or dust particles containing an infectious agent. Infectious microorganisms from an infected person or animal may be carried in this manner and widely dispersed by air currents within a room or over a long distance, and they may thereby become inhaled or deposited on a susceptible host.

As used herein, the phrase “airborne precautions” refers to special air handling and ventilation required to prevent airborne transmission. People coming in contact with a patient who has a disease which is known or suspected to be transmitted by airborne transmission are advised by the Centers for Disease Control to wear respiratory protection such as disposable surgical masks or, most preferably, an N95 respirator.

As used herein, the term “aerosol” refers to a gaseous suspension of solid and/or liquid particles.

As used herein, the term “animal” refers to any living organism that possesses a respiratory system with a respiratory tract that includes a trachea and lungs, such as, mammals, birds, and reptiles.

As used herein, the words “communicable” and “contagious” refer to a type of disease which is transmitted from one person to another either directly, by contact with discharges from the body; or indirectly, via substances or inanimate objects.

As used herein, the term 4“contaminant” refers to a chemical agent or biological organism/pathogen that can potentially harm a human being or animal; the term “contamination” refers to the act or process of contaminating.

As used herein, “diagnostic” means something that is used to make a diagnosis, i.e., the identification of one or more pathogens by a scientific evaluation of physical signs, symptoms, history, laboratory test results, and/or procedures. A diagnostic may include a device which is not intended for definitive diagnosis, but rather is a screening type of device which is capable of providing useful information such as that which would be used to select for further testing to obtain a definitive diagnosis.

As used herein, the phrase “droplets” refers to pathogenic microorganisms suspended in large-particle droplets (larger than 5 μm in size) of fluid containing pathogenic microorganisms. Droplets may be generated when a subject who is infected with a clinical disease or is a carrier of a disease is exhaling, coughing, sneezing or talking. The droplets may then be transmitted by contact with the conjunctivae or mucous membranes of the nose or mouth of a susceptible host. The microorganisms within the droplets may thereby be inhaled by the host and cause infection. Droplets may become aerosolized and are then referred to as “airborne droplet nuclei”. Examples of diseases which are spread by droplets include, but are not limited to, chickenpox, common cold, influenza, measles and mumps.

As used herein, the phrase “droplet infection” refers to an infection acquired by the inhalation of droplets.

As used herein, the phrase “droplet precautions” refers to safeguards designed to reduce the risk of droplet transmission of infectious agents. These precautions apply to any patient known or suspected to be infected with epidemiologically important pathogens that can be transmitted by infectious droplets. Large-particle droplet transmission requires close contact between source and recipient persons because droplets do not remain suspended in the air and generally travel distances of 3 feet or less. Therefore, special air handling and ventilation (i.e., “airborne precautions”) are not required to prevent droplet transmission, However, the Center for Disease Control advises that people who are within 3 feet of a patient who has a disease which is transmitted by droplet transmission wear a mask (for example, a surgical mask) to prevent contamination.

As used herein, the term “expiratory” pertains to exhalation from the lungs.

As used herein, “expired” refers to any gas and/or particulate matter that has been expired from the lungs of a subject.

As used herein, the term “fluid” refers to any gas, liquid, or mixture of gas and liquid; various types of aerosols and particulate matter may be entrained with such fluids.

As used herein, “infectious” refers to a disease which is caused by the invasion of the body by pathogenic microorganisms, and which is capable of being transmitted between subjects by infection, with or without actual contact between subjects.

As used herein, the phrase “face masks” refers to any personal protective device that is worn on the face, covers at least the nose and mouth, and is used to reduce the wearer's risk of inhaling hazardous airborne particles (including dust particles and infectious agents), gases, or vapors. The many types of masks available include (1) particulate respirators, which filter out airborne particles; (2) “gas masks,” which filter out chemicals and gases; (3) airline respirators, which use compressed air from a remote source; (4) self-contained breathing apparatus, which include their own air supply; (5) fold-flat surgical masks; and (6) molded, cup-shaped masks.

As used herein, “filtering” refers to a type of face mask which includes either a filtering device to protect the wearer of the mask from exposure to external communicable pathogens, a filtering device to protect other persons from exposure to communicable pathogens potentially expired by the wearer, or both.

As used herein, the term “pathogen” refers to an agent that causes diseases, including, but not limited to a living microorganism, such as, a bacterium, a fungus, a virus, a viroid, prions/proteins, and so forth.

As used herein, the term “N-95 respirator” refers to one of nine types of disposable particulate respirators. Particulate respirators are also known as “air-purifying respirators” because they protect by filtering particles out of the air as a person breathes. These respirators protect only against particles—not gases or vapors. Since airborne biological agents such as bacteria or viruses are particles, they can be filtered by particulate respirators.

As used herein, the term “surgical mask” refers to a disposable mask that will provide barrier protection against droplets.

As used herein, the term “unilateral” refers to a type of expiratory droplet collection strip which is inserted through only one side of a wearer's face mask. In comparison, a “bilateral” expiratory droplet collection strip may be incorporated or inserted all the way through a face mask so that it extends beyond both sides of the mask.

These terms may be defined with additional language in the remaining portions of the specification.

### Expiratory Droplet Collection Strip

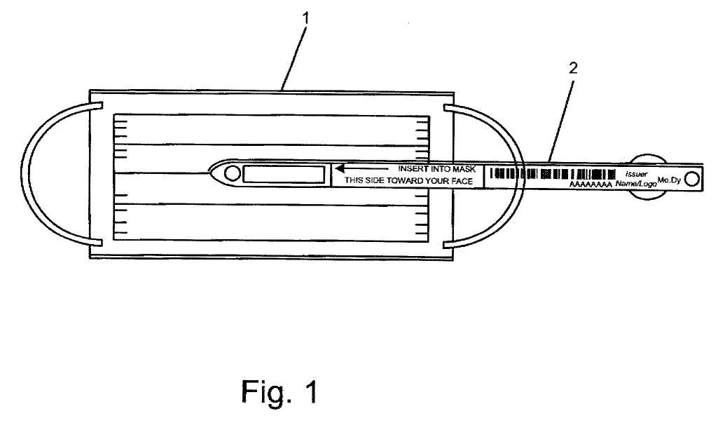

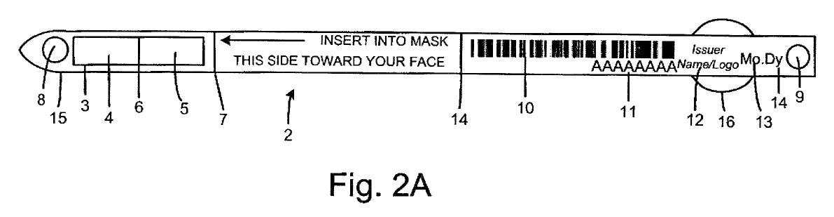

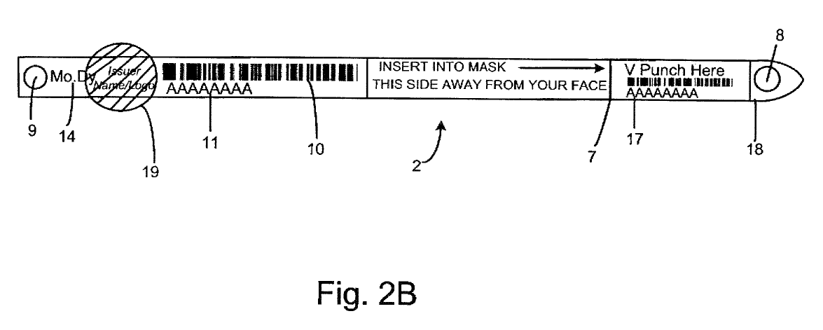

The present invention comprises an expiratory droplet collection strip capable of being inserted or fitted between the wearer's mouth and the interior of the face mask. FIG. 1 is a rear view of a fold-flat, filtering face mask 1 that demonstrates the appropriate positioning of a unilateral expiratory droplet collection strip 2 within said face mask 1 prior to donning of the mask. FIG. 2A is a front view example of a unilateral expiratory droplet collection strip 2. FIG. 2B is a rear view example of a unilateral expiratory droplet collection strip 2. In one embodiment of the invention, expiratory droplet collection strips may be conveniently attached to the interior side of the face mask prior to donning the mask by means of an adhesive contained on the strip or mask.

In another embodiment of the invention, the expiratory droplet collection strips may be inserted between the user's mouth and the face mask after the face mask is donned and while it is being worn by the user. As shown in FIG. 2A, 15 is a rounded edge of the strip which facilitates insertion between the skin of the cheek and the face mask while the face mask is already in place. The strips have the advantage of being adaptable to a wide variety of face mask types, including, but not limited to, fold-flat surgical masks and molded, cup-shaped, disposable respirators. Furthermore, the strips can be quickly and conveniently inserted into the face masks of wearers while the face mask is being worn, thereby avoiding the risks of exposure to the wearer that would otherwise be associated with the removal or substitution of a face mask already in place. In this way, expiratory droplet collection strips can be distributed and fitted to wearers in areas of congregation and other high traffic environments having a high risk of exposure to airborne pathogens. In a preferred embodiment of the invention, the strip is flat and bendable so that it may be inserted between the wearer's mask and cheek and slid into place so as to properly position the collection means area 3 of the device over the user's mouth area.

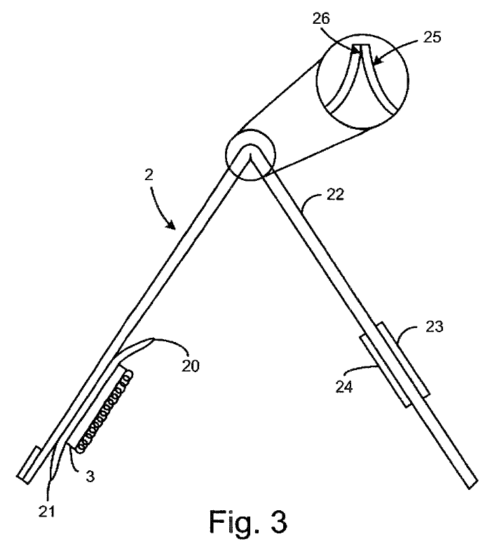

FIG. 3 is a cross sectional view of a semi-folded unilateral expiratory droplet collection strip 2 that contains a polyester or other suitable fibrous collection material that is affixed to the collection area 3 of the strip. Tabs 20 and 21 are useful for the removal of the non-permanent adhesively mounted detachable collection material units. Such units would be uniquely identified on their opposite sides (not shown). The strip material 22 is a flexible paper or plastic of sufficient thickness to permit insertion of the strip into the face mask of a wearer that is already in place. 23 and 24 are side views of an adhesive label placed to permit the adhesion of the strip to the wearer's face or if alternatively worn in a folded position, to be affixed to the exterior portion of the face mask. 25 shows an alternative embodiment of the invention which is a folded version that utilizes two separate materials affixed at a fixation point 26 so as to create a tension between the two materials and thereby clip to the edge of a face mask to assist in holding the strip proper position during wear.

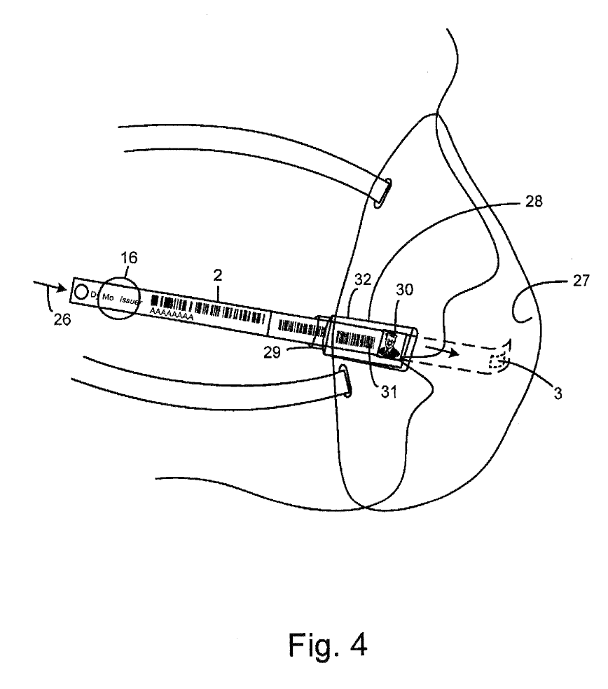

FIG. 4 shows a side view of a molded, cup-shaped mask 27 with the proper positioning of an unfolded unilateral expiratory droplet collection strip 2 following its insertion in the direction indicated by the arrow 26 between the facial skin and interior portion of a wearer's face mask 27. Correctly positioned, the collection area 3 of the expiratory droplet collection strip affixed to the side of the strip facing the wearer's face and is positioned directly in front of the wearer's mouth to capture and collected exhaled pathogens within the interior portion of the face mask 27.

The strip of the present invention may incorporate a means to hold or fasten the collection strip in place once inserted into the mask so as to maintain its proper positioning and avoid interference with movements of the wearer's mouth and jaw. In one embodiment, the fasting means is an adhesive that is attached or applied to the collection strip. As shown in FIG. 2B, 18 is an adhesive and/or adhesive under a disposable paper that may be optionally utilized by the wearer to adhere the strip to the interior of a wearer's face mask so as to maintain the proper positioning of the collection area over the wearer's mouth area and avoid interference with movements of the wearer's mouth and jaw. The adhesive 18 may be affixed to the area of the strip 2 opposite to the collection area 3 so as to adhere to the interior of the face mask over the user's mouth. In another embodiment of the invention, the fastening means may comprise a non-adhesive material or spiny device capable of loosely attaching to the interior fabric of the mask so as to resist movement of the strip once in place and in contact with the interior of the mask. In another embodiment of the invention, the fastening means may comprise a non-allergenic adhesive capable of adhering to the skin of wearer, such as the cheek skin, ear area or chin, thereby holding the collection strip in place. In a further embodiment of the invention, the fastening means could include a clip, fold or adhesive designed to attach to the edge of the face mask. FIG. 4 shows a reusable spring clip or fastener 28 that clips to the edge of the wearer's face mask and permits insertion of disposable strips through an insertion slit 29 mounted or incorporated within such clip or fastener.

In another embodiment of the invention, the fastening is accomplished by means of a fold in the collection strip whereby the exterior portion of the strip extending outside the face mask can be folded over and attached to the exterior surface of the face mask. In lieu of a fold, a separate layer of material may be attached to the exterior end of the of the collection strip so that once the strip is separated and inserted it extends over the exterior face of the face mask, thereby providing the necessary tension to attach to the face mask in a “clothespin” style. In a further embodiment of the invention, the strip is of sufficient length so as to extend out the opposite side of the face mask upon insertion, such as between the cheek and face mask on the opposite side of the wearer's face. Such strips have the advantage of being held in place at two points in which the face mask makes contact with the user's face. Such strips also have the added advantage of being externally visible, readable and collectable on both sides of the wearer's face. Such strips can be fairly easily threaded through the opposite side with minimal assistance from or by the wearer. In the case of such bilateral design, the collection area would ideally be located towards the center of the collection strip, as opposed to towards one of the proximal ends as in the case of the “unilateral” strips previously described by the inventor. In further embodiments, the collection strip may be designed to adhere to the interior portion of the mask over the wearer's mouth area, adhere to the wearer's skin on one or both sides of the face, or further comprise a fold or clothespin design to extend and/or adhere to the exterior portion of the collection strip over the exterior portion of the face mask. In a further embodiment, such a bilateral collection strip may be of sufficient length so as to bring the opposite ends in contact and capable of fastening or adhering to each other by means of an adhesive or other means such as a cohesive material that binds only with itself.

In another embodiment of the invention, a fastening means that adheres to the skin of the wearer so as to be externally visible also incorporates a liquid crystal, colorimetric temperature assay at the point of contact with the skin so as to potentially identify wearers having an elevated skin temperature indicative of a fever. As shown in FIG. 2A, 16 is an optional adhesive sticker or adhesive calorimetric thermometer such as a Biodot to adhere the strip to the cheek skin of the wearer so as to maintain the proper positioning of the collection area over the wearer's mouth area and serve as an indicator of a suspected feverish condition of the wearer. As shown in FIG. 2B, a transparent adhesive sticker or calorimetric assay 19 is placed to adhere to the cheek skin of the wearer. FIG. 4 shows an adhesive label or colorimetric thermometer 16 that adheres to the wearer's cheek, thereby holding the strip in place. Such examples are not intended to preclude the incorporation of any and all assays into the original manufacture of the actual face masks, or to preclude the subsequent addition of such devices to the masks.

In order to maintain reusability and life of any face mask, a fastener may be separate from the collection strips and attached to the face mask to allow for the quick insertion, removal, and reinsertion of multiple expiratory droplet collection strips with little or no additional effort required to correctly position and maintain each additional collection strip in place. Such fasteners could also incorporate externally evident useful information about the wearer. As shown in FIG. 4, a reusable clip or fastener may contain information useful to identify the wearer, such as a photo or digital image of the wearer 30 as well as the name, sex, age, height and weight information, and the time and place of original issuance of the strip. The addition of this information can reduce the potential for quarantined or potentially contagious persons to escape detection by assuming the identity of others through the wearing of expiratory droplet collection devices issued to other persons. As shown in FIG. 4, the reusable fastener/badge may also have a bar code 31 or other machine readable information and/or a unique radio frequency identification (RFID) tag 32 imbedded within it. A radio frequency identification tag may provide a rapid electronic means to associate a wearer and trace them to the bar coded or otherwise uniquely identified expiratory droplet collection strips at the point and time of collection of the strips. In this way, expiratory droplet collection strips that subsequently test positive for the contagious disease of interest may be immediately traced back to the original wearer. Once identified, an individual's unique bar coded or machine readable identification number, such as in the case of an RFID, can be utilized to identify, contact, test and potentially quarantine infectious individuals as they subsequently pass through various protective congregative checkpoints established to protect other members of the population.

### Expiratory Droplet Collection Material

A wide variety of material is suitable for the collection of respired airborne pathogens and may be incorporated into the expiratory droplet collection strips of the present invention. Examples include, but are not limited to, materials traditionally utilized in nasal or throat swabs, including polyester, Dacron, and cotton, as well as other fibrous materials including paper, Tyvek and plastics. For example, polyester is currently recommended by the World Health Organization (WHO) for use in nasal and throat swabs for the collection of the Avian influenza virus (H5N1). Filtering paper and other breathable materials traditionally utilized in the production of protective respiratory face masks may also be suitable for collection and testing of used, discarded face masks, such face masks that may be collected upon exit of a hospital setting by a healthcare worker, patient or visitor. Collection materials may also be impregnated with substances useful for the storage and transport of acquired samples, including, but not limited to, antibiotics and antifungals such as gentamicin sulfate and amphotericin B, as well as dried veal infusion broth, dried albumin fraction V, Stuart's media or other preservatives and commonly used virus transport mediums. Ideally, any such additives should be incorporated in a fashion so as to avoid contact with the wearer.

In an embodiment of the invention, samples of the used collection material may be obtained by one or more hole punchers or other means and may be distinguished from face masks and swabs described in the prior art by virtue of the placement or printing of identifiers unique to each mask or strip in the nose/mouth area that are useful for subsequent identification, tracking and association with punched samples. As shown in FIG. 2A, the collection area 3 may be directly incorporated into the expiratory droplet collection strip or alternatively may comprise a suitable fibrous collection material such as polyester that is affixed to the expiratory droplet collection strip. 4 and 5 are two collection areas that may be separated by means of one or more perforations (6 and 7) in the collection material or strip to facilitate separate testing of area 4 in a pooled group of samples while preserving the other collection area 5 and/or the entire strip for storage and subsequent re-sampling on an individual basis. 8 and 9 are punch holes useful for the automated collection, storage, transport and processing of expiratory droplet collection strips as a whole or only the collection areas 4 and 5 of the strips. 10 is a pre-printed or labeled machine-readable bar code that is useful to identify the strip to track and associate collected strips to the person from whom the sample was obtained. 11 is the same identifier number as 10 but is in an alpha numeric format. 12 is a color-coded name and/or logo of the agency, hospital or other issuer of the strip. 13 and 14 are month and date information to that may be pre-printed or labeled as well as individually color-coded to signify the month and day that such strip is intended to be used or deemed expired. As shown in FIG. 2B, 17 is an additional printed bar code and alphanumeric unique identifier printed immediately opposite to one or more detachable collection areas to assist in the tracking of collection areas once detached from the strip.

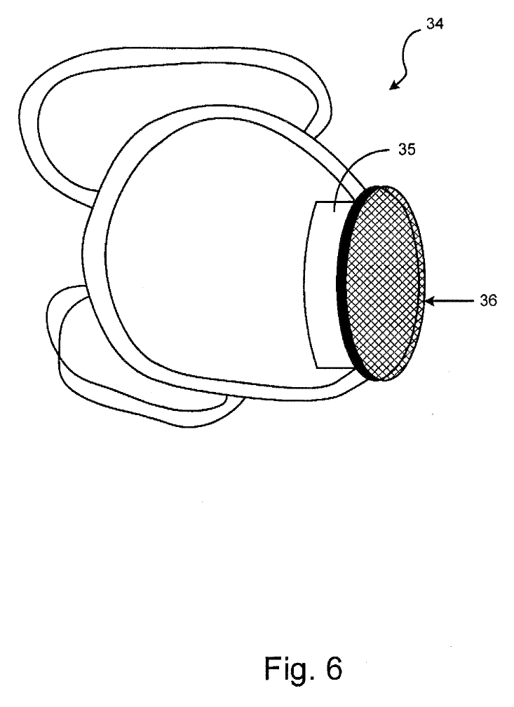

As opposed to commonly used nasal or throat swabs, the collection area of the expiratory droplet collection strips of the present invention for use with respiratory face masks will ideally utilize a larger surface area and will be positioned externally in front of the nose and mouth of the wearer and inside the interior of the wearer's face mask. Such collection materials are held in place for a period of time which is substantially longer than the amount of time required to obtain a nasal or throat swab. In an alternative embodiment of the invention, expiratory droplet collection materials may be positioned on the external portion of respiratory face masks which comprise a one-way exhaust valve, such as certain disposable respirators which are commonly used as dust masks and are known in the art. In the case of face masks which comprise an exhaust valve, it is anticipated that in addition to the collection material, additional filtering material would be fitted over the exterior portion of the exhaust valve so as to protect others from transmission of airborne pathogens by the wearer. Expiratory droplet collection materials may be either attached to the interior or the exterior of the valve. In one embodiment, the additional filtering material may be in the form of a detachable cartridge that may be attached to the exhaust valve. FIG. 6 is a perspective view of a molded, cup-shaped respiratory mask 34 which is fitted with an exhaust valve 35 having a detachable droplet collection device and exhaust filtering cartridge 36.

### Sterility and Packaging

The present invention comprises designs and methods to maintain sterility so as to protect the wearer from cross infection by others as well as to preserve the integrity of the assay which will ultimately be performed for diagnostic and/or screening purposes. Accordingly, in one embodiment of the present invention, expiratory droplet collection strips are incorporated within individual sterile packages such as paper or foil until ready for use by the wearer. As shown in FIG. 2A, 14 is a fold which allows the expiratory droplet collection strip to be folded on itself to preserve the sterility of the interiorly folded side of the strip containing the sterile collection and to limit exposure to the potentially contaminated, exteriorly folded surface areas of the strip. In another embodiment of the invention, the expiratory droplet collection strip may be sealed in a sterile, one-time use, disposable wrapper. The wrapper may be transparent to facilitate reading of the strip. The external packaging may further comprise a unique bar code or other machine readable identification such as an RFID which matches with that of the expiratory droplet collection strip contained within to assist in the tracking and association of distributed expiratory droplet collection strips. In a further embodiment of the invention, the external packaging may serve the additional purpose of providing the user with a means to store and transport expiratory droplet collection strips and to limit contamination and cross-contamination after use. In a further embodiment, the expiratory droplet collection strips are packaged or contained with or within a re-closable test tube. Such packaging or test tube may also contain the liquid reagents necessary for subsequent storage and transport of expiratory droplet collection strip samples. In a still further embodiment of the invention, the packaging, test tube or kit may also contain a separate lateral flow, pass through or other immunoassay specific for one or more pathogens of interest. Packaging and collection strips may incorporate tamper evident and other features to prevent misuse, including, but not limited to, pre-printed markers and inks indicative of contact with liquid prior to collection or assay, and/or incorporated positive controls upon assay.

### Devices and Methods for Collection of Used Masks

The present invention comprises devices useful for the mass collection of face masks which comprise the expiratory droplet collection strip of the present invention. In one embodiment of the invention, used or discarded face masks are turned in and collected, for example upon egress outdoors or from a confined or enclosed area of congregation, such as a hospital, mass transit system, airline flight, airport or office building. Preferably, each face mask is separately bagged by the wearer prior to collection to prevent cross contamination between masks.

In a further embodiment of the invention, each face mask contains a unique identifier capable of being re-traced to the individual wearer and is collected in a biohazard suitable bin. One or more sharp tipped hollow metal rods or bits are used to puncture and punch samples from a number of used face masks contained within such bin. The rod or bit containing the samples is then thoroughly flushed and or washed with suitable transport and storage media suitable for one or more viruses or bacteria of interest. The flushed liquid may then be assayed by polymerase chain reaction (PCR), for example. In the event that one or a group of such bin samples tests positive for the pathogen of interest, then individual bins, and then individual face masks from such bin, may be individually tested until one or more infectious face masks are identified. The unique identifier associated with the face mask may then be utilized to trace the identity of the wearer.

### Devices and Methods for Collection of Used Strips

While the previously described methodologies related to the collection of used masks are inventive and useful, their potential limitations in application under pandemic conditions can be predicted and overcome with further embodiments of the present invention. First, direct sampling of used face masks as described would require that the used face masks first be removed, thereby potentially exposing wearers and other persons to the infectious disease which they are intended to guard against. Similar risks would also accompany any nasal or throat swabbing screening procedures since such invasive procedures would be difficult to accomplish without additional risk to a subject who is already donning a protective face mask. Second, wearers may also be exposed at the time and place of initial distribution and donning of such traceable face masks. Accordingly, persons may prefer to don face masks in private at home, prior to going out in public. This would make distribution of traceable face masks a logistical problem and may requiring wearers to substitute traceable face masks for non-traceable ones once they are out in public, thereby risking exposure to the user at the time and place of substitution. In the case of airline travel and a potential global pandemic, persons or animals could conceivably be arriving from any nation or city in the world, making uniform application difficult or impossible. Furthermore, there are likely to be practical limitations associated with the timely production and distribution of traceable face masks made by different manufacturers at a time when global face masks will be in greatest demand. Finally, collection of entire face masks is cumbersome and it is difficult to obtain consistent samples from masks through the sampling methodology described above due to variations in the conditions of bins.

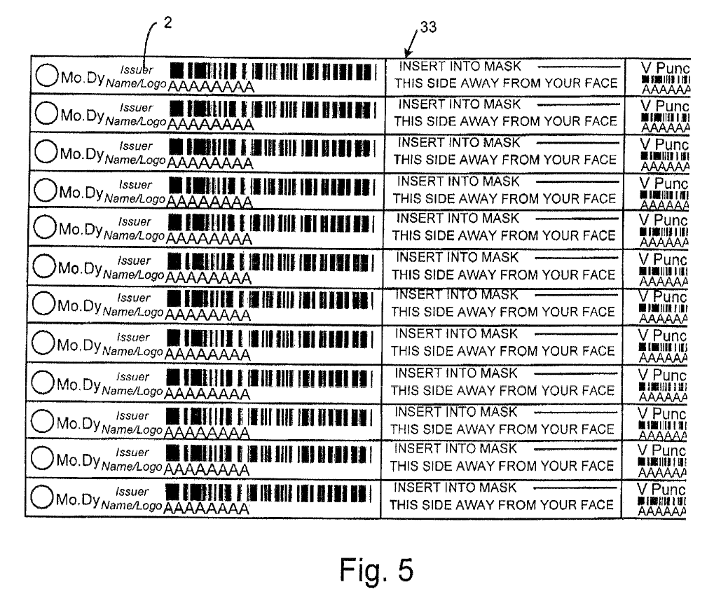

Accordingly, the present invention in one aspect comprises expiratory droplet collection strips which may be sold or distributed separately and used in association with any type of respiratory face mask. FIG. 5 shows a sheet 33 of uniquely identified, computer-generated, mass-produced, preprinted, bar coded, expiratory droplet collection strips that represents a significant manufacturing step to facilitate the rapid manufacture of uniquely printed expiratory droplet collection strips on demand prior to the steps of cutting, folding, individual packaging and sterilization. The expiratory droplet collection strips as further described herein offer the advantage of ease of collection from wearers without requiring wearers to remove their protective face masks. Accordingly, in various embodiments of the invention such strips may be incorporated into the masks at the time of original manufacture, retrofitted to already manufactured face masks, or distributed as a kit in individually wrapped sterile bags containing a face mask and one or more expiratory droplet collection strips and fasteners. Preferably, expiratory droplet collection strips are distributed in individually wrapped sterile packages that are opened by the wearer immediately prior to use. The expiratory droplet collection strips as further described herein permit their easy insertion into almost any face mask of any type or manufacture in manner that permits the sterile collection area to reach the external mouth area within any face mask without requiring the wearer to remove a face mask that may already be in place. Similarly, expiratory droplet collection strips may be subsequently later slid out and collected without requiring the wearer to remove the protective face masks. By utilizing a flexible thin strip design, expiratory droplet collection strips will only interfere with the interface between the wearer's face and the face mask while the strip is in place. Respiratory droplet collection strips as described also have the important advantage of permitting the direct printing and labeling of useful human and machine readable information which may be viewed on the externally visible portion of the strip while the strip is in place. Such information may include, but is not limited to, an alphanumeric identifier, bar code, issuer name, year, month, and date, each of which may utilize a combination of various color-coded inks as to be easily evident at a distance. The information on the external portion of the strip may further comprise anti-forgery printing of visible and/or ultraviolet visible inks and holograms. The external area may also include an area to obtain the fingerprint of the wearer immediately prior to placement or at the time of collection or both to validate the identity of the wearer. Expiratory droplet collection strips may utilize a separate, reusable, externally visible, adjustable fastener or clip capable of attaching to the expiratory droplet collection strip and to the wearer's face mask so as to maintain the collection area of the strip in proper position over the wearer's mouth. Preferably, such reusable fastener or clip can also serve as a badge incorporating a photo and description of the intended wearer and/or radio frequency identifier.

In a preferred embodiment of the invention, the expiratory droplet collection strips contain multiple separate or detachable collection areas for the collection and transport of samples. The purpose of having multiple collection areas is to facilitate screening of pooled samples, whereby at least one detachable collection area is collected and transported for rapid detection by being pooled with up to a multitude of other samples for assaying or screening by polymerase chain reaction (PCR), for example. One or more other samples are individually collected and stored under refrigerated and/or frozen conditions for later individual sampling. In the event that a pathogen of interest is detected in a pooled sample, than the separate, individually stored samples comprising the group of such pool may be individually tested to identify the one or more infectious persons. Such pooled screening approaches would be expected to greatly minimize assaying requirements by only requiring individual testing on groups of samples known to contain at least one positive test outcome, as opposed to requiring each individual sample within the group to be tested. Samples can be collected and tested in a hierarchy of groups, subgroups and individual basis to reduce the required time and complexity of unnecessary screening in which entire groups or subgroup populations are negative for the contagious disease of interest.

The present invention also comprises collection devices for the collection of respiratory assay strips. Strips may contain pre-punched holes on either end to hold, coordinate and automate the collection and testing process. The strips may also incorporate metallic, magnetic materials. Collected strips may be designed for testing by PCR or other means on an automated basis. Strips or collection areas may be collected into rolls for automated processing, testing and storage and recall. In a preferred embodiment, a handheld collection device is capable of wirelessly reading the bar code of the strip as well as obtaining wearer information from the wearer's badge or other identification, such as by a magnetic swipe of a driver's license or credit card.

### Software

The present invention also comprises software, preferably web-based software and databases to collect relevant information to record the distribution of individual uniquely identified expiratory droplet collection strips so as to provide a means to trace a positive sample back to the associated wearer. A central database may be maintained for this purpose as well as for providing the ability to identify and track the movement of individuals considered to be at risk and of risk and therefore subjected to further screening and/or quarantine. The software and database can also be extremely useful to assist in international, national, state and local epidemiological and bio-surveillance efforts in order to determine and track disease and rapidly gauge the success or failure of various alternative containment strategies.

### RF Tags and RFIDs

In one embodiment of the present invention, the face masks and/or collection strips incorporate radio frequency tags (“RF tags”) or radio frequency identification devices (“RFIDs”) to assist in the collection of expiratory droplet collection samples and therefore also assist in tracking and monitoring the compliance of individual wearers. An RF tag contained within an assay strip or face mask can provide an audible or other electronic means to screen persons as they walk through a radio frequency detection field or gate. RF tag systems are currently widely used by retail stores to control shoplifting and in inventory control.

RFID tags and labels have a combination of antennas and analog and/or digital electronics, which may include, for example, communications electronics, data memory, and control logic. RFID tags and labels are widely used to associate an object with an identification code. For example, RFID tags are used in conjunction with security-locks in cars, for access control to buildings, and for tracking inventory and parcels. Some examples of RFID tags and labels appear in U.S. Pat. Nos. 6,107,920, 6,206,292, and 6,262,292, all of which are incorporated herein by reference.

RFID tags and labels include active tags, which include a power source, and passive tags and labels, which do not include a power source. In the case of passive tags, in order to retrieve the information from the chip, a “base station” or “reader” sends an excitation signal to the RFID tag or label. The excitation signal energizes the tag or label, and the RFID circuitry transmits the stored information back to the reader. The reader receives and decodes the information from the RFID tag. In general, RFID tags can retain and transmit enough information to uniquely identify individuals, packages, inventory and the like. RFID tags and labels also can be characterized as to those to which information is written only once (although the information may be read repeatedly), and those to which information may be written during use. For example, RFID tags may store environmental data (that may be detected by an associated sensor), logistical histories, state data, etc.

An RF tag incorporated into a expiratory droplet collection strip or device can be used to identify persons that are not wearing a expiratory droplet collection device such as a expiratory droplet collection strip or face mask as they walk through a radio frequency detector, by virtue of a lack of an audible or other electronic signal thus alerting personnel to a potential non-compliance situation. Alternatively, a positive or audible RF signal could alert the wearer and/or collection personnel to a situation in which an expiratory droplet collection device has failed to be removed and collected. A radio frequency identification device or RFID can be useful in identifying an individual wearer or discriminating between different groups or populations based upon place and time of issuance, for example. A unique RFID could be used as an efficient means to associate a particular person with an expiratory droplet collection strip collected from a wearer at the same time.

Currently, surveillance and security systems using radio frequency identification (RFID) tags or other radio frequency (RF) tracking systems can track the movement and location of objects and people that are wearing the tags within the margin of error for the device. However, these systems have no means of tracking people or objects that are not wearing the RF tracking tags. Similarly, some sophisticated video surveillance systems can detect and monitor the movement and location of objects within a given area. However, these video surveillance systems require a human interface to determine if the object or person should or should not be in a specific area. It is desirable to resolve these problems of monitoring an area with video surveillance and determining if an object or person is authorized to be in that area.

U.S. patent application Ser. No. 10/748,455, incorporated herein by reference, describes the use of RFID tags that can be associated with a wide variety of various biosensors that may be potentially associated with a wide variety of reusable or disposable items commonly found in a hospital environment.

U.S. Pat. No. 6,987,451, incorporated herein by reference, describes a system for monitoring an area with surveillance system and determining if a person or object is authorized to be in that area by correlating the location of objects being tracked by the surveillance system (such as, but not limited to, digital, non-digital and infra-red video) with those of a tracking system (such as a RF tracking system or similar devices like, but not limited to, a GPS and radio signal transceiver system). The system outputs a list of objects that are being monitored within a given area by the surveillance systems, and whether or not these objects are associated with the tracking tags, such as a RF tracking tag, being tracked by the monitoring and tracking systems. The system can then notify the system operator of an unauthorized object or person within a given area.

U.S. patent application serial number 11/040,496, incorporated herein by reference, describes the use of RFID tags for tracking and monitoring visitors to improve the current process of visitor identification through ID badges, stickers, etc., and therefore allows for much more than granting limited access in the context of a healthcare setting. Such patent application also describes the concept of an engaged system that does more than simply record events by quickly alerting security systems if a visitor badge enters an unauthorized area.

Neither U.S. patent application Ser. No. 10/748,455, U.S. Pat. No. 6,987,451 nor U.S. patent application Ser. No. 11/040,496, neither individually, nor taken together, describe RFID methods or systems for the purpose of sampling and collecting respiratory droplets from persons or animals or environments in order to detect potential infectivity of airborne communicable diseases, or to improve compliance in connection with infection control procedures requiring the use of personal protective equipment (such as, face masks, gloves, shields or gowns that may incorporate RFID technology) nor to reduce the transmission of airborne communicable diseases through the enforcement of quarantine procedures.

An object of the present invention is to provide methods and devices for collecting respiratory droplets, improve compliance with infection control procedures and assist in the enforcement of quarantine procedures by utilizing personal protective equipment such as, but not limited to, face masks or face mask-associated badges and devices, such as, respiratory droplet collection strips that are associated with RFID tags. In addition, an object of the present invention also includes a droplet collection device that contains one or more RFID tags to assist in monitoring its origination and tracking through multiple read write steps as it proceeds through the collection, assay and storage process to improve the ability to determine the original source of droplet collection devices that upon subsequent assay, are determined to be either positive for the pathogen(s) of interest or of interest.

In one embodiment of the present invention, the RFID tag transmissions are automated such that the RFID tag transmits data collected from the droplet collection strip and/or other devices on the mask without the need for human intervention, to a data aggregation center, for example a center for disease control, or public health agency for epidemiological surveillance and disease reporting purposes.

In one embodiment of the present invention, the RFID tag transmissions occur among and between mask wearers as a network of interconnected mask wearers that transmit data by packet switching. These networks can be combined with any other type of device network that supports the same protocol, including but not limited to using the standard Internet Protocol (IP). Wherein the data from one mask is transmitted to user of a mask within range is a “network of networks” that consists of potentially thousands of smaller networks of mask wearers, which together carry various information, such as disease status, about individual wearers.

### Thermometer Device

In one embodiment of the present invention, the face mask incorporates a diagnostic device capable of identifying potentially infectious individuals by means of a thermometer which is capable of identifying individual wearers having body temperature in excess of the average normal range (i.e., 98.6 degrees F.). The additional thermometer device may take a variety of forms.

In a preferred embodiment, the thermometer may comprise an inexpensive colorchange, photochromic or thermochromic film or paint attached to the mask that is in contact with the facial skin of the wearer, such as the cheek or the bridge of the nose. Persons suspected of fever may be rapidly and easily identified from a group by persons trained to recognize colors indicative of abnormal elevated body temperature.

In a further preferred embodiment, the thermometer could measure the temperature of the exhalation of the user and need not be in contact with the skin of the wearer. In a still further embodiment, the thermometer could be applied as a colormetric paint, such as a colormetric paint available from Edmund Scientific, New York, N.Y. The paint may be applied to a portion of the mask, the color of which would estimate body temperature and be evident to an observer competent in recognizing the relevance of certain colors as they relate to normal and feverish conditions.

In another preferred embodiment, expiratory droplet collection assays and identification systems can be utilized in conjunction with electronic external thermosensing devices, such as an ear thermometer or thermoimaging camera to identify and select potentially contagious individuals. Once identified on the basis of temperature or other potential signs of illness, such persons may be selected for on-the-spot screening. Under such circumstances, expiratory droplet collection strips that have been previously inserted elsewhere have the important advantage of not requiring individuals to risk exposure by the immediate removal of their respiratory face masks in order to obtain sample specimens for testing. By virtue of having exhaled for a substantial amount of time on the expiratory droplet collection strips, an adequate sample suitable for assaying may be easily obtained by the simple removal of the expiratory droplet collection strips with the suspected contagious individual being requested to wait until results are obtained. If such results prove positive, a person may be further selected for confirmatory nasal swabs and/or blood tests, which if also prove positive result in potential quarantine and treatment.

### Biosampling Device

In another embodiment of the present invention, the face mask incorporates a diagnostic device capable of identifying potentially infectious individuals and diseases by means of a biosampling material or apparatus that is in direct communication with the oral and/or nasal expiratory flow of the wearer and is capable of collecting infectious particles expired by the wearer. Such biosampling material may comprise a designated area on the interior of the mask anticipated to be in direct communication with the expiratory flow of the user without the need to add additional special biosampling material, provided that the filtering material comprising the mask is capable of collecting biosamples for later analysis. In a further preferred embodiment, the exterior or interior of the biosampling area contains unique preprinted identifiers such as serial numbers or machine readable bar codes and other relevant information, for example, to assist in the later identification of the wearer in the event that later bioanalysis of the material tests positive for the disease of interest. By locating the preprinted serial numbers within the biosampling area of interest, required collection and handling can be dramatically reduced and standardized for further testing by a punch system, for example, that punches and collects only the relevant biosampling area, allowing the remainder of the mask to be discarded without loss of relevant information regarding the wearer. Relevant information may or may not include not only information regarding the wearer, but also time and place of issue, thereby providing relevant information regarding the transit and potential spread of disease.

In a preferred embodiment, the biosampling material may comprise a uniquely identifiable preprinted material or strip separate from the mask that is capable of being affixed to the interior of a wide variety of face masks. Such material should be affixed in a fashion as to avoid the potential for inhalation by the wearer while still allowing convenient removal, collection and uniformity to facilitate rapid automated testing and analysis.

In another embodiment of the invention, face masks may include a device indicative of the length of time of time of wear. One way to accomplish this would be to package the face mask in a sealed aluminum or other atmospherically impervious material utilizing an inert gas purge, such as nitrogen or argon, immediately prior to sealing. The exterior of the mask could contain a mark of lettering comprised of an oxidizable substance or ink that changes color or lettering in a time-dependent manner. Incorporation of such an indicator could serve to identify wearers required to return used masks in exchange for newly issued ones, thereby facilitating bio-sampling, as well as serving as a reference by which to accurately quantify relative rates of potential since viral counts measured in bio-samples would increase in a linear fashion based upon time of wear by the user.

In a further preferred embodiment, the face mask may contain a translucent material in front of the mouth so as to be externally visible for observation or noninvasive biosampling without the need to remove the face mask. In a further embodiment, the face mask having a translucent front may be non-invasively sampled by means of a device that is capable of non-invasive detection of infectious diseases such as a light emitter directed at the translucent bio-sampling material in combination with a spectroscopic light detector capable of detecting wavelengths reflected by the bio-sample indicative of one or more the infectious diseases or disease processes.

In a further preferred embodiment of the invention, the face mask may incorporate a filter to protect the wearer from exposure to external airborne communicable pathogens and one-way exhalation valve. The exhalation valve would serve to not only aid to the comfort of the wearer by reducing the temperature and humidity within the mask, but also serve the important function of providing a means by which to direct and concentrate expiration of the wearer towards bioassays and additional potential devices in a consistent and more potent manner. Furthermore, use of an exhalation valve could also serve the important purpose of transmitting expired particles and gases from the interior of the mask to collection points exterior to the mask, thereby facilitating collection and analysis without requiring removal of the mask.

In a further preferred embodiment of the invention, the face mask may contain an external cartridge in interface with the exhalation valve, wherein the external cartridge contains a filtering mechanism to prevent exposure to others of infectious particles exhaled by the wearer. In another embodiment, the external cartridge may contain a bio-sampling material capable of collecting exhaled particles of the wearer for further analysis upon removal of such the cartridge. In a further embodiment of the invention, the external cartridge contains a migration or other immunoassay device capable of immediately identifying and characterizing exhaled particles of the wearer that selectively bind to antibodies or other proteins selective for one or more particular infectious diseases of interest. Such an immunoassay may utilize a reservoir of water or other liquid attached to the cartridge that may be punctured or tapped so as to form a solution containing the exhaled particles of the wearer for analysis by migration immunoassay. In a further embodiment, the cartridges may be color coded or labeled as to coincide with days or other periods in which they should be used, as opposed to being turned in to authorities.

The present invention further comprises a device capable of capturing and storing biologic fluids, such as, respired pathogens, saliva, sputum, red or white blood serum, respiratory gases, and urine. In a further embodiment, the device can also collect biometric information by electronic or other means (heart rate, blood pressure, body temperature, etc.). The devices may be useful whether distributed either before or after a suspected outbreak. The device may represent an inexpensive collection device that can be rapidly read and interpreted in the event of an outbreak. Such inexpensive devices may be made in a machine readable, uniform format that can be sequentially analyzed, e.g., via robotic, PCR, ELISA, HPLC, immunoassay, capillaripheresis, or 2D or 3D gel electrophoresis. The device may represent an advanced electronic apparatus capable of direct or local analysis, as well as remote communication to any center, including a center for disease control, for example.

Approximate time and date information for specimen capture can be evidenced utilizing a non-electronic, predictably degrading/transforming chemical initiated upon contact with specimen (i.e. use). The device(s) may also be capable of sequentially capturing additional information so as to serve as an individual log of progression or lack thereof. Such device can be designed to be tamper-proof, such as a tamper-evident seal if the device were designed as bracelet, for example. In addition, on-site validation may be compared through the immediate analysis of a new saliva sample, for example as compared to prior samples to confirm authenticity.

In an embodiment of the invention, the device may be incorporated into a partial or full face mask (utilizing light weight paper or other particle retaining material). Such respiratory mask serves the dual purpose to protect the individual from air-borne pathogens. It may resemble a commonly used paper or other breathable material used to cover the mouth and nose, for example. The mask would also contain, however, material suitable for the collection and entrapment of biologic or other samples exhaled through or around such collection strip or port. Such collection material may be detachable from the mask or simply represent an outlined area that is machine-readable. The collection device may contain a preservative to preserve living organisms, such as bacteria or viruses, or it may contain a safe disinfectant to deactivate biologic organisms. Face masks may be mandated (for public transportation, for example), with new masks issued regularly. Such masks may contain a reactive biologic or other agent that may be visible to the naked eye or by electronic means so as to identify potentially contagious persons. Such masks may also contain bar codes or other specific identifiers that can at a minimum track their original issuance (by time, place, or even by individual). New masks may be issued only upon return of a previously issued mask, for example, to aid in compliance. It may also include a tamper evident device to indicate whether it was removed since placement. The masks may be packaged in material containing little or no air, or packaged under modified atmospheric conditions, such as, nitrogen or argon purge. Once opened, an externally evident chemical or other marker reactive with normal atmospheric conditions can estimate and make evident the hours of use, such as through a gradual color change or disappearing timeline such as that utilized estimate the remaining life of disposable batteries, for example.

In an embodiment of the invention, the devices may be supplied in an inexpensive yet uniform form that is machine readable, e.g., by PCR, ELISA, HPLC, hand held field sampler, immunoassay, capillary transit, 2D or 3D gel electrophoresis, or other means. In another embodiment, the devices may incorporate an electronic or other means to communicate its status remotely as via a USB connection or via telephone jack.

In another embodiment of the present invention, the information obtained from the newly invented device may be used to rapidly prescribe alternative existing or investigational therapeutic interventions with the advantage of rapidly identifying homogeneous groups at baseline, as well as consistently and rapidly retrieving important surrogate and actual markers of disease activity so as to rapidly optimize therapeutic and other intervention strategies.

The collection device aspect of the invention also has the important advantage of aiding in and can be useful for identifying pathogens of interest by providing a uniform and consistent means to collect numbers of viral, bacterial or other pathogens from severe/advanced, moderate and newly infected/mild to asymptomatic individuals.

In another embodiment of the present invention, the device may contain one or more saliva collecting probes, one or more needles to collect blood samples, one or more urine collection sticks, and/or feces sampler, as well as the wearable face mask.

1-2. (canceled)

3: A face mask that comprises (a) a filter to protect the wearer from exposure to external airborne communicable pathogens; (b) a one-way exhalation valve; and (c) a removable external cartridge that interfaces with an externally accessible side of the exhalation valve, wherein the cartridge contains a filter to protect the public from exposure to airborne communicable pathogens potentially expired by the wearer.

4: The mask of claim 3
wherein the cartridge is made at least in part of a transparent or partially transparent material.

5: A device for the identification of persons potentially infected with a communicable disease, comprising a filtering face mask and a migration immunoassay device.

6: The device of claim 5
wherein a liquid necessary for a migration immunoassay is collected in a sealed reservoir located inside the mask.

7: A face mask that comprises (a) a filter to protect the wearer of the mask from exposure to external airborne communicable pathogens; (b) a filter to protect other persons from exposure to airborne communicable pathogens potentially expired by the wearer; and (c) at least one diagnostic device for identifying potentially infectious individuals.

8: The face mask of claim 7,
wherein the diagnostic device is a thermometer.

9: A device capable of non-invasive detection of infectious diseases of interest that comprises a (1) light emitter directed at the translucent biosampling material of claim 6, and (2) a spectroscopic light detector means capable of detecting wavelengths reflected by the biosample indicative of an infectious disease or disease process.

10: A filtering face mask that comprises (a) a mask body; and (b) a device that is capable of collecting droplets expired by a wearer of the mask.

11-12. (canceled)

13: A method to track an outbreak of a disease comprising (a) the issuance of face masks which comprise a device which identifies whether a wearer of the mask is infected with a contagious disease; (b) collection of the face masks; (e) obtaining a sample from the face masks; and (d) testing samples to determine whether the wearer of the face mask is infected with said disease.

14: A method to improve quarantine procedures which comprises issuance of face masks containing externally visible personal identification information.

15-16. (canceled)

17: An expiratory droplet collection device for incorporation into a face mask.

18: The device of claim 17,
wherein the device is a strip.

19. A method of use of the device of claim 17 that comprises inserting the device between a face mask and a cheek of a wearer without removal of the face mask from the wearer.

20: A method of use of the device of claim 64, which comprises exposing an external side of the device comprising the adhesive and using the adhesive to adhere the device to the interior of a face mask body.

21: A method of manufacturing of the strip of claim 18 which comprises (1) using software to create a unique label for the expiratory droplet collecting strip; (2) using software to print a sheet of said strips; (3) attaching adhesive to said sheet; (4) attaching expiratory droplet collection material to said sheet; and (5) cutting said sheet into individual strips.

22: A cartridge that interfaces with the externally accessible side of an exhalation valve of a face mask, wherein the cartridge comprises a filter to protect the public from exposure to airborne communicable pathogens potentially expired by the wearer.

23. (canceled)

24: A method of detecting individuals suspected of fever comprising use of at least one thermoimaging camera directed at a population of people.

25: The method of claim 24, wherein the thermoimaging cameras are placed at various angles at the same group of subjects and analyzed by computer to account for differences in distance as well as walking or running speed of subjects.

26: The mask of claim 3, wherein the cartridge contains at least one diagnostic device to identify potentially infectious individuals.

27: The mask of claim 26, wherein the diagnostic device is selected from the group consisting of a thermometer, a cough event device, a biosampling material which is aligned with the exhalation valve of the mask and that is capable of entrapping expiratory pathogens of the wearer; and a migration immunoassay strip coated with one or more groups of antibodies specific for one or more pathogens of interest.

28: The mask of claim 3, wherein the cartridge is labeled with identifying information.

29: The mask of claim 3, wherein the cartridge is labeled with an externally visible color coded label or mark to aid in rapid identification of cartridges requiring replacement or unique handling.

30: The mask of claim 3, wherein the cartridge is printed or labeled externally with a substance that once exposed to normal atmospheric conditions, oxidizes or otherwise changes colors in a manner indicative of time of wear.

31: The device of claim 5, wherein a liquid necessary for a migration immunoassay is collected from vapor exhaled by a wearer of the mask via a collection device located inside the mask.

32: The device of claim 5, wherein the mask includes a collection device that utilizes a desiccant to collect vapor, wherein the desiccant preferably is calcium chloride.

33: The device of claim 5, wherein the mask includes a collection device that comprises a condenser, wherein the condenser preferably is formed of a thermally conductive material having one side exposed to the exterior atmosphere and a condensing side exposed to an interior to the mask so as to reduce the temperature of the material and facilitate condensation on the side of the material exposed to the interior of the mask.

34: The device of claim 5, wherein the migration immunoassay migrates from an interior of the mask to the exterior of the mask for immediate observation or collection without the need to remove or disrupt the protective function of the mask.

35: The device of claim 5, wherein the migration takes place in a material having a gradient from wet to dry.

36: The device of claim 5, wherein the migration takes place by capillary absorption.

37: The device of claim 5, wherein the migration takes place by positive air pressure through a material having a gradient of high air pressure towards a lower air pressure, wherein the gradient corresponds to a desired direction of migration.

38: The device of claim 5, wherein the migration takes place by lateral capillary flow.

39: The device of claim 5, wherein the migration takes place by lateral capillary flow takes place by a radially oriented capillary flow from a center of a reservoir upon tap.

40: The device of claim 5, wherein the migration takes place under a force of gravity.

41: The face mask of claim 8, wherein the thermometer is selected from the group consisting of a digital device, a color change device, a photochromic device, a thermochromic device, and a thermally expansive material that acts as an indicator of temperature.

42: The face mask of claim 8, wherein the diagnostic device is a thermometer in contact with the skin of the wearer.

43: The face mask of claim 8, wherein the diagnostic device is a thermometer incorporated into the mask for contact with the skin of a wearer at a point of interface between the skin of the wearer and the mask.

44: The face mask of claim 43, wherein the thermometer is located to contact the skin of the wearer at the face-mask interface at the bridge of the nose.

45: The face mask of claim 7, wherein the diagnostic device is a thermometer in contact with respiratory expiration of the wearer.

46: The face mask of claim 7, wherein the diagnostic device is a biosampling material affixed to an interior of the mask, and wherein the material is capable of entrapping pathogens expired by the wearer.

47: The face mask of claim 7, wherein the diagnostic device is a biosampling material which comprises essentially the entire body of the filtering face mask.

48: The face mask of claim 7, wherein the mask is labeled with identifying information.

49: The face mask of claim 7, wherein the mask is labeled with information which identifies a wearer of the mask.

50: The face mask of claim 7, wherein the mask is labeled with information which identifies a time and place of issuance of the mask, or a time and place of collection of the mask.

51: The face mask of claim 7, wherein the mask further comprises a removable sticker imprinted with identifying information.

52: The face mask of claim 7, wherein the mask is labeled with an alphanumeric serial number or bar code.

53: The face mask of claim 7, wherein the mask is labeled with an externally visible color-coded mark, wherein the mark identifies face masks which require replacement or unique handling.

54: The face mask of claim 7, wherein the mask is labeled externally with a substance that changes color, wherein a change in color indicates the amount of time that the mask has been worn.

55: The face mask of claim 54, wherein the substance is one which changes color due to oxidation, upon exposure to normal atmospheric conditions.

56: The face mask of claim 7, wherein the mask is labeled with identifying information located on a relevant biosampling area of the mask.

57: The face mask of claim 56, wherein the diagnostic device is biosampling area which corresponds to the areas of the nostrils and mouth.

58: The face mask of claim 7, wherein the diagnostic device is a biosampling area which is physically separated from the body of the face mask to facilitate collection and handling of biosamples without losing identification information.

59: The face mask of claim 7, wherein the diagnostic device is a biosampling material which is a different material than the material used to form the body of the mask, and wherein the biosampling material is affixed to the interior of the mask where it is capable of being conveniently removed following use.

60: The face mask of claim 7, wherein the diagnostic device is a biosampling material affixed to the interior of the mask by an adhesive.

61: The face mask of claim 7, wherein the diagnostic device is a biosampling material affixed to the interior of the mask externally visible via a cutout in the face mask located approximately in front of the mouth and nose, wherein the cutout is permanently sealed with a translucent material to permit external viewing of an externally facing side of the biosampling material, and wherein the interior side of the translucent material preferably comprises the biosampling material.

62: The face mask of claim 7, wherein the diagnostic device is a biosampling material having an externally facing side which contains identifying information.

63: The device of claim 17, wherein the device comprises a collection material.

64: The device of claim 17, wherein the device comprises a strip of adhesive material.

65: The device of claim 17, wherein the strip is folded.

66: The device of claim 17, wherein the strip is inserted into a face mask in a unilateral fashion.

67: The device of claim 17, wherein the strip is inserted into a face mask in a bilateral fashion.

68: The device of claim 17, wherein the strip carries pre-printed identifying information.

69: The device of claim 17, wherein the device utilizes radio frequency identification technology to store identifying information.
